(19) United States
(12) Patent Application Publication
(10) Pub. No.: US 2009/0215978 A1
(43) Pub. Date: Aug. 27, 2009

(54) PROCESS

(75) Inventors: Simon Darby Roberts, London (GB); Stephan William Colley, London (GB)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: Davy Process Technology Limited, London (GB)

(21) Appl. No.: 11/817,126

(22) PCT Filed: Feb. 21, 2006
(86) PCT No.: PCT/EP2006/060156

§ 371 Date: Oct. 7, 2008

(30) Foreign Application Priority Data
Feb. 28, 2005 (GB) 05040795

(51) Int. Cl. C08G 63/189; C07C 43/20; C07C 43/205; C07C 67/39; C08G 63/181; C07C 63/46; C07C 63/15

(52) U.S. Cl. 528/298; 568/660; 568/659; 568/662; 560/77; 560/100; 528/271; 562/480; 562/490

(57) ABSTRACT

A process for the alkoxyalkylation of an unsubstituted aromatic substrate or a substituted aromatic substrate, said substitution being via a carbon-carbon bond, said process comprising reacting the aromatic substrate with a dialkoxyalkane in the presence of a catalyst.

The present invention relates to a process for alkoxyalkylation of an aromatic substrate. More particularly it relates to a process for the selective alkoxyalkylation of an aromatic substrate. Still more particularly, it relates to a process for the selective alkoxymethylation of an aromatic substrate. In particular it relates to the dialkoxymethylation of an aromatic substrate or the monoalkoxymethylation of a mono-substituted aromatic substrate. Most particularly it relates to the alkoxymethylation of naphthalene or a β-substituted naphthalene to afford a 2,6-disubstituted naphthalene. In a preferred arrangement, the present invention relates to a process for the production of 2,6-di(alkoxymethyl)naphthalene. In a second embodiment the present invention relates to a process for the production of polymers and in particular polyesters such as polyethylene naphthalate.

It is well known that alkylated polycyclics such as 2,6-dialkylnaphthalenes are valuable materials as they are precursors to polyesters such as polyethylene naphthalate. Polyethylene naphthalate has superior strength and barrier properties over other polyesters, such as polyethylene terephthalate, and is used to make fibres, films and packaging materials. In view of the improved properties obtained with polyethylene naphthalate substantial research effort has been carried out with a view to obtaining an economical manufacturing route to the product and/or its precursors.

Polyethylene naphthalate is generally produced either by esterification of 2,6-naphthalenedicarboxylic acid or by transesterification of dimethyl 2,6-naphthalenedicarboxylate. The 2,6-naphthalenedicarboxylic acid and 2,6-naphthalenedicarboxylate starting materials for these reactions are generally produced from 2,6-dialkylnaphthalene.

However, the production of the desired 2,6-dialkylnaphthalene starting materials has particular difficulties since there are ten dialkylnaphthalene isomers.

There are a number of known methods for the production of 2,6-dimethylnaphthalene. For example, as described in U.S. Pat. No. 4,963,248, 2,6-dimethylnaphthalene can be recovered from fractions during kerosene reformation or, as described in European Chemical News, P. 30, 28 Sep. 1992 and in U.S. Pat. No. 6,121,501, it can be recovered from fractions of FCC oil. Whilst these processes provide the desired compound, because of the small differences in the boiling points of the various dimethylnaphthalene isomers complicated selective adsorption, separation and crystallisation procedures have to be used. The separation of the 2,6- and 2,7-isomers are particularly difficult. These isomers can also form a eutectic mixture.

It has also been suggested that isomerisation technology can be used to convert mixed dimethylnaphthalene isomers into the desired 2,6-dimethylnaphthalene. Examples of potentially suitable isomerisation technologies are described in U.S. Pat. No. 4,777,312, U.S. Pat. No. 5,495,060, U.S. Pat. No. 6,015,930 and U.S. Pat. No. 6,018,087. However as described in U.S. Pat. No. 6,057,487 only the 1,5- and 1,6-isomers are readily isomerised.

Effort has also focussed on selectively producing the 2,6-isomer or the di-isomer ‘triad’ (i.e. 2,6-, 1,5- and 1,6-dimethylnaphthalene). In particular, efforts have been made to provide processes which avoid the co-production of the 2,7-isomer. One such process involves a series of reactions starting from o-xylene and butadiene to prepare 1,5-dimethylnaphthalene which is further isomerised to the desired isomer over a zeolite catalyst. Suitable processes are described in U.S. Pat. No. 4,990,717, U.S. Pat. No. 5,073,670, U.S. Pat. No. 5,118,892, U.S. Pat. No. 5,030,781 and U.S. Pat. No. 5,012,024. However, this four step route is far from ideal since a number of secondary reactions occur which necessitates purification steps to be carried out for each intermediate.

A more direct process to 2,6-dimethylnaphthalene has been described in U.S. Pat. No. 4,795,847. This process starts from naphthalene or methylnaphthalene and comprises methylation with a suitable methylating agent in the presence of a zeolite catalyst. Zeolite catalysts have been shown to hold a number of advantages over homogeneous Friedel-Crafts type catalysts. These benefits include the ease of catalyst separation, waste, corrosion and toxicity minimisation, as well as the ability to affect selectivity. As reported in U.S. Pat. No. 5,001,295 the selection of the appropriate zeolite for the methylation of naphthalene or 2-methylnaphthalene can produce a mixture that is rich in the desired 2,6-isomers. However due to the near identical molecular diameters of the 2,6- and 2,7-dimethylnaphthalene isomers the product mixture only weakly favours the desired isomer.

Efforts have been made to enrich 2,6-dimethylnaphthalene in the product by transalkylating unwanted dimethylnaphthalene isomers with naphthalene over a zeolite to produce 2-methylnaphthalene which can then be selectively alkylated to 2,6-dimethylnaphthalene. One process of this type is described in U.S. Pat. No. 6,011,190. Further isomerisation to 2,6-dimethylnaphthalene then occurs followed by separation from the remaining dimethylnaphthalene isomers and other products.

To utilise the shape-selective properties of zeolites, bulkier substituents have been added to the naphthalene substrate such that the difference in the critical molecular dimension between the 2,6- and 2,7-isomer is maximised. As described in WO 90/03960, U.S. Pat. No. 5,900,519 and U.S. Pat. No. 4,950,824, isopropylation of naphthalene or 2-isopropylnaphthalene over a zeolite catalyst can produce mixtures rich in 2,6-diisopropylnaphthalene.

Substantial research effort has been made to improve the 2,6/2,7 ratio by careful optimisation of the catalyst properties and process conditions. A number of alternative substituents have been attached to naphthalene and its derivatives, although none thus far have met the desired criteria for selectivity, ease of subsequent oxidation (generally the bulkier the alkyl substituent the more difficult the oxidation), and atom efficiency. In this connection it is noted that four carbon atoms would be lost during oxidation of 2,6-diisopropylnaphthalene to 2,6-naphthalenedicarboxylic acid. Examples of processes using alternative substituents include that described in U.S. Pat. No. 4,873,386 in which an ethyl substituent is used, that described in Org. Biomol. Chem., 2003, 1552-1559 in which the substituent used is tert-butyl and that described in U.S. Pat. No. 5,210,350 in which dicyclohexyl is used. U.S. Pat. No. 5,210,355, U.S. Pat. No. 5,235,115 and U.S. Pat. No. 5,321,182 describe processes in which combinations of substituents are used.

Even once the production of the 2,6-dialkylnaphthalene starting material has been achieved, problems are encountered in the oxidation, purification, esterification and/or further refinement to high-purity 2,6-naphthalenedicarboxylic acid or 2,6-naphthalenedicarboxylate.

One method for the production of 2,6-naphthalenedicarboxylic acid is described in U.S. Pat. No. 3,870,754, U.S. Pat. No. 3,856,855, U.S. Pat. No. 4,709,088, U.S. Pat. No. 4,716,245, U.S. Pat. No. 4,794,195, U.S. Pat. No. 4,950,786, U.S. Pat. No. 5,144,066, U.S. Pat. No. 5,183,933 and WO 04/015003. In this process the 2,6-dialkylnaphthalenes are oxidised in acetic acid in the presence of a cobalt/manganese catalyst and a bromide promoter. However, due to the corrosive nature of the chemicals used in this reaction, it is necessary to fabricate the reactor vessels from high cost materials such as titanium. A further disadvantage of the process is that 2,6-naphthalenedicarboxylic acid obtained requires substantial purification to remove the various impurities. These impurities include trimellitic acid, 6-formyl-2-naphthoic acid and brominated naphthalene compounds. In addition, insoluble heavy metal complexes (cobalt and manganese) are formed, particularly with trimellitic acid, which can result in downstream process fouling. The purification of 2,6-naphthalenedicarboxylic acid is further complicated as the acid has low solubility in most solvents and decomposes at its melting point.

Crude 2,6-naphthalenedicarboxylic acid may be esterified to 2,6-naphthalenedicarboxylate with methanol as purification of the ester, whilst still complex, offers certain advantages. Examples of esterification processes can be found in U.S. Pat. No. 6,013,831 and U.S. Pat. No. 6,211,398. U.S. Pat. No. 5,095,135 and U.S. Pat. No. 5,254,719 teach that sulphuric acid is an effective catalyst for the esterification reaction and reacts with the heavy metal impurities to form soluble sulphate salts. However, corrosion issues and waste sulphate disposal are further problems encountered with this process.

In order to achieve high-purity 2,6-naphthalenedicarboxylate, dissolution in a suitable solvent, typically an aromatic hydrocarbon, and further insolubles separation is required, followed by recrystallisation and distillation steps. Thus the six common steps required to convert 2,6-dialkyl naphthalene to 2,6-naphthalenedicarboxylate which is suitable for polyethylene naphthalate production are 1) oxidation 2) esterification 3) dissolution 4) separation 5) recrystallisation and 6) distillation. A further process step involves the recovery and recycling of the expensive oxidation catalyst metals.

It is therefore desirable to provide a process which provides a 2,6-disubstituted naphthalene which can readily be converted to polyethylene naphthalate. This can be achieved where at least one of the substituents on the naphthalene is an alkoxyalkyl, particularly an alkoxymethyl, group. 2,6-di(alkoxymethyl)naphthalene offers significant advantages over 2,6-dialkylnaphthalene in the production of polyester as it is more readily oxidised, thus milder conditions can be employed in the oxidation step of the polyester production process. The use of milder conditions will overcome the problems of low yield and the requirement for expensive purification steps which are noted with prior art processes.

It is also desirable to provide a process for the selective alkoxyalkylation of other aromatic substrates.

Thus according to the present invention there is provided a process for alkoxyalkylation of an unsubstituted aromatic substrate or a substituted aromatic substrate, said substitution being via a carbon-carbon bond, said process comprising reacting the aromatic substrate with a dialkoxyalkane in the presence of a catalyst.

The alkoxyalkylation is preferably a process selective for a desired substitution. The selectivity may be achieved by any suitable means. In one arrangement, a shape selective catalyst such as a zeolite may be used.

Where the catalyst is a zeolite any suitable zeolite may be used. The choice of zeolite may influence the product selectivity as the geometry of the framework type will affect the position of aromatic substitution. The reaction can be catalysed over a broad acidity range. Wholly acidic zeolites and materials partially exchanged with basic cations may be used. Zeolite mordenite has been found to offer particular advantages.

The SiO₂/Al₂O₃ ratio of the zeolite may influence activity and selectivity. It is believed that acidic mordenite catalysts having higher SiO₂/Al₂O₃ ratios offer improved selectivity for the desired isomer.

Any suitable physical form of catalyst may be used. Thus powders and extrudates may be used.

In an alternative arrangement a non-shape selective catalyst may be used in a shape selective environment. For example a homogenous catalyst such as a Lewis acid catalyst may be used if held within a shape selective host. Any suitable Lewis acid catalyst may be used. Suitable catalysts include AlCl₃.

The aromatic substrate may be any substrate that is susceptible to electrophilic substitution. The aromatic substrate may be a mono or polycyclic aromatic compound and will preferably be a mono or polycyclic hydrocarbon although heteroaryl compounds may also be used. Any suitable heteroaryl compound may be used.

Where the substrate is a polycyclic aromatic compound it may have fused rings or rings that are connected via a bond, for example a bi-phenyl.

The aromatic substrate may be substituted or unsubstituted. Where it is substituted, it will generally be mono-substituted. Where mono-substituted aromatic substrates are used, any suitable substituent may be used. The substituent will generally facilitate the second substitution occurring at the desired position and/or facilitate downstream processing. To facilitate downstream processing it may be desirable to use a substituent which is in a suitable oxidation state or readily oxidisable state for transformation into a polyester precursor. Suitable substituent groups include ester groups, methyl groups and acid groups.

The process of the present invention is particularly suitable for the production of a di-substituted aromatic substrate either by adding a second substituent to a mono-substituted aromatic substrate or by di-substitution of an unsubstituted aromatic substrate.

Where the substrate is mono-substituted, the position of the mono-substitution will be selected to provide the desired product.

In a particularly preferred arrangement, the disubstituted product is substituted such that the substituents are spaced to the maximum amount. For example, where the aromatic substrate on which the reaction is carried out is a monocyclic aromatic compound, the substituents will be located in the para position. Similarly, where the aromatic substrate is a polycyclic compound they will be spaced to the greatest extent. Thus where the substrate is naphthalene, the substituents will be located at the 2,6 position. Where the substrate is a biphenyl, the substituents will generally be located at the 4,4′ position.

Where the product of the process of the present invention is for use as a polyethylene naphthalate precursor, the aromatic substituent will preferably be naphthalene or it may be a mono-substituted naphthalene. Where a mono-substituted naphthalene is used, the substituent will generally be an easily oxidised substituent and will generally be attached to the naphthalene at the 2-position. Thus suitable substrates will include 2-(alkoxyalkyl)naphthalene, alkyl-2-naphthoate, 2-naphthoic acid or 2 alkylnaphthalene. Suitable 2-(alkoxyalkyl)naphthalenes include 2-(alkoxymethyl)naphthalene.

Any suitable dialkoxyalkane may be used. Dialkoxymethanes are particularly preferred. Suitable dialkoxymethanes include dimethoxymethane and diethoxymethane. The alkoxymethylating agent may be chosen to influence the product selectivity. Without wishing to be bound by any theory, it is believed that where a bulkier substituent is formed, selectivity will be improved.

The reaction may be carried out in the liquid phase or in the gas phase. The process may be operated as a batch or a continuous process. The reaction may be carried out in a fixed bed reactor, a fluidised bed reactor or a slurry reactor. Whatever type of reactor is used, the process may be carried out in the presence of a shape selective catalyst or host.

A liquid-phase continuous process utilising a fixed bed of catalyst or of shape selective host offers some advantages. It will be understood that where the catalyst is shape selective, the shape selective host will generally be the catalyst.

The reaction may be carried out in the presence or absence of solvent. Suitable solvents include non-polar hydrocarbons such as hexane or cyclohexane. Acetone, trichloromethane or diethyl ether may also be used. If no solvent is used, the reactants may act as solvent.

Any suitable temperature may be used. It is desirable to use a temperature which provides a good yield of substituted product whilst minimising by-product formation. It is believed that if the temperature is too low, the yield of the reaction is low and if the temperature is too high, the yield of desired product is diminished due to competing side reactions or the further reaction of the product to undesirables.

Temperatures in the range of from about 100° C. to about 200° C. are useful with temperatures of from about 110° C. to 170° C. being preferred.

The process of the present invention may be carried out under self-generated pressure which is typically less than about 150 psig. This is particularly suitable where the process is carried out as a batch process. Pressures of from about 5 barg to about 50 barg may offer certain advantages.

Any suitable reaction time may be used. It is believed that if the reaction time is too short the yield of desirable product may be reduced. However, if the reaction time is too long, the selectivity may be reduced as by-products, undesirable isomers, and breakdown products may be obtained. Reaction times in the region of from about 1 hour to about 72 hours may be used. Preferably the reaction time is from about 1 hour to about 40 hours with times in the region of from about 4 hours to about 18 hours being preferred.

According to a second aspect of the present invention there is provided a process for the production of a carboxylic acid or carboxylate comprising:
producing a di(alkoxyalkyl)aromatic compound or a substituted-mono(alkoxyalkyl) aromatic in accordance with the above first aspect of the present invention; and
oxidising the alkoxyalkyl side chains to the corresponding carboxylic acid or carboxylate.

According to a third aspect of the present invention there is provided a process for the production of polyesters comprising polymerising the carboxylic acid or carboxylate produced by the process of the above second aspect.

Where the aromatic compound is naphthalene, the process will produce polyethylene naphthalate.

The process may also be used for the production of other polyesters. The process of the third aspect of the present invention may be used to produce polyalkylene terephthalates, polyalkylene phthalates, polyalkylene naphthalates, and polyalkene biphenylates.

Thus, where the aromatic substrate is benzene or a mono substituted benzene, the process may produce polyethylene terephthalate. Similarly, biphenyl or a mono-substituted biphenyl, particularly one substituted at the 4 position can be used in the process to provide a route to specialty polyesters which are based on a biphenyl backbone.

This process offers an improved process since the substituent groups are in a favourable oxidation state prior to the oxidation step and thus milder oxidation conditions can be employed.

The present invention will now be described with reference to the following examples. Unless stated, all examples were carried out in 100 ml stirred autoclaves, operating under self-generated pressure.

In the examples below, the numbers expressed as percentages have been rounded to the nearest whole number, except numbers which are less than one which are expressed to 1 decimal place. Isomer ratios are direct comparisons of gas (chromatograph peak areas and expressed to 1 decimal place.

### EXAMPLE 1

The methoxymethylation of 10 mmol naphthalene is carried out with 80 mmol dimethoxymethane over 4 g H+ zeolite catalyst with 60 ml cyclohexane as solvent.

The catalyst used is Beta SiO₂/Al₂O₃ 25. The reaction was carried out at 150° C. After 18 hours there was found to be 59 mol % naphthalene conversion, giving yields of 37 mol % (methoxymethyl)naphthalene and 6 mol % di(methoxymethyl)naphthalene and <0.1 mol % of the undesirable by-product methylene-bis-naphthalenes. The 2/1 ratio was 1.9 and the 2,6/2,7 ratio was 2.9.

### EXAMPLE 2

Example 1 was repeated with the catalyst being replaced with a sodium exchanged beta zeolite SiO₂/Al₂O₃ 25. After 18 hours there was found to be 46 mmol % naphthalene conversion, giving yields of 18 mol % (methoxymethyl)naphthalene and 5 mol % di(methoxymethyl)naphthalene and <0.1 mol % of the undesirable by-product methylene-bis-naphthalenes. The 2/1 ratio was 1.6 and the 2,6/2,7 substitution ratio was 6.0.

### EXAMPLE 3

Example 1 was repeated with the catalyst being replaced with a acidic zeolite mordenite SiO₂/Al₂O₃ 20. After 18 hours there was found to be 13 mol % naphthalene conversion, giving yields of 4 mol % (methoxymethyl)naphthalene and 0 mol % di(methoxymethyl)naphthalene and <0.1 mol % of the undesirable by-product methylene-bis-naphthalenes. The 2/1 ratio was 12.9.

### EXAMPLES 4 TO 8

Example 1 was repeated utilising different solvents. The results are set out in Table 1. In each example, 60 ml of the solvent was used.

TABLE 1

| Example No | 4 | 5 | 6 | 7 | 8 |
|---|---|---|---|---|---|
| Solvent | Cyclohexane | Hexane | Acetone | Trichloro- | Diethyl- |
| | | | | methane | ether |
| Naphthalene conversion | 59 | 47 | 52 | 54 | 37 |
| (mol %) | | | | | |
| (methoxymethyl)naphthalene | 37 | 23 | 39 | 37 | 8 |
| (mol %) | | | | | |
| 2/1 ratio | 1.9 | 1.8 | 2.5 | 2.6 | 3.5 |
| di(methoxymethyl)naphthalene | 6 | 1 | 6 | 3 | 1 |
| (mol %) | | | | | |
| 2,6/2,7 ratio | 2.9 | 2,6- only | 5.2 | 3.7 | 2,6- only |

### EXAMPLES 9 TO 13

Example 1 was repeated at a variety of temperatures. The results are set out in Table 2.

TABLE 2

| Example No: | 9 | 10 | 11 | 12 | 13 |
|---|---|---|---|---|---|
| Temperature (° C.) | 115 | 120 | 125 | 130 | 150 |
| naphthalene conversion | 36 | 34 | 47 | 54 | 59 |
| (mol %) | | | | | |
| (methoxymethyl)naphthalene | 19 | 20 | 20 | 18 | 37 |
| (mol %) | | | | | |
| 2/1 ratio | 2.9 | 3 | 2.6 | 2.1 | 1.9 |
| di(methoxymethyl)naphthalene | 2 | 2 | 2 | 3 | 6 |
| (mol %) | | | | | |
| 2,6/2,7 ratio | 2.7 | 2.4 | 2.7 | 2.0 | 2.9 |

### EXAMPLES 14 TO 19

Example 1 was repeated at a temperature of 120° C. and at a range of reaction times. The results are set out in Table 3.

TABLE 3

| Example No | 14 | 15 | 16 | 17 | 18 | 19 |
|---|---|---|---|---|---|---|
| Time (hours) | 4 | 6 | 9 | 18 | 36 | 72 |
| naphthalene conversion | 41 | 45 | 46 | 44 | 46 | 51 |
| (mol %) | | | | | | |
| (methoxymethyl)naphthalene | 18 | 16 | 20 | 20 | 21 | 21 |
| (mol %) | | | | | | |
| 2/1 ratio | 2.6 | 3 | 2.5 | 3 | 2.5 | 2.2 |
| di(methoxymethyl)aaphthalene | 1 | 2 | 2 | 2 | 2 | 2 |
| (mol %) | | | | | | |
| 2,6/2,7 ratio | 2,6- only | 2.1 | 2.5 | 2.4 | 2.0 | 1.5 |

### EXAMPLES 20 TO 23

Example 1 was repeated at a temperature of 120° C. and at a reaction time of 4 hours with varying amounts of catalyst. The results are set out in Table 4.

TABLE 4

| Example No | 20 | 21 | 22 | 23 |
|---|---|---|---|---|
| Amount of catalyst (g) | 1 | 2 | 4 | 8 |
| Naphthalene conversion | 22 | 27 | 41 | 58 |
| (mol %) | | | | |
| (methoxymethyl)naphthalene | 17 | 16 | 18 | 18 |
| (mol %) | | | | |
| 2/1 ratio | 3.2 | 2.8 | 2.6 | 2.7 |
| di(methoxymethyl)naphthalene | 0 | 0 | 1 | 5 |
| (mol %) | | | | |
| 2,6/2,7 ratio | — | — | 2,6- only | 7.3 |

Whilst selectivity was noted to be low with a low amount of catalyst, it is believed that longer reaction time would give the desired product. Without wishing to be bound by any theory, it is believed that in the short time period and low catalyst amount, only mono substitution occurs.

### EXAMPLES 24 AND 25

Example 1 was repeated for a reaction time of 4 hours and at a temperature of 120° C. with 758 mmol dimethoxymethane and with a catalyst having differing SiO₂/Al₂O₃ ratios. The results are set out in Table 5,

TABLE 5

| Example No | 24 | 25 |
|---|---|---|
| Catalyst | Beta SiO₂/ | Beta SiO₂/ |
| | Al₂O₃ 25 | Al₂O₃ 75 |
| Naphthalene conversion | 82 | 67 |
| (mol %) | | |
| (methoxymethyl)naphthalene | 38 | 34 |
| (mol %) | | |
| 2/1 ratio | 2.2 | 1.7 |
| Di(methoxymethyl)naphthalene | 18 | 14 |
| (mol %) | | |
| 2,6/2,7 ratio | 5.8 | 5.0 |

### EXAMPLES 26 AND 27

Examples 24 and 25 were repeated with a mordenite catalyst having differing SiO₂/Al₂O₃ ratios. The results are set out in Table 6.

TABLE 6

| Example No | 26 | 27 |
|---|---|---|
| Catalyst | Mordenite SiO₂/ | Mordenite SiO₂/ |
| | Al₂O₃ 20 | Al₂O₃ 90 |
| naphthalene conversion | 52 | 86 |
| (mol %) | | |
| (methoxymethyl)naphthalene | 40 | 44 |
| (mol %) | | |
| 2/1 ratio | 3.7 | 4.0 |
| di(methoxymethyl)naphthalene | 8 | 28 |
| (mol %) | | |
| 2,6/2,7 ratio | 11.6 | 11.7 |

### EXAMPLE 28

Example 27 was repeated with the dimethoxymethane being replaced with 535 mmol diethoxymethane as reagent. The results are compared in Table 7.

TABLE 7

| Example No | 27 | 28 |
|---|---|---|
| Reagent | dimethoxymethane | diethoxymethane |
| | 758 mmol, 67 ml | 535 mmol, 67 ml |
| naphthalene conversion | 86 | 27 |
| (mol %) | | |
| (alkoxymethyl)naphthalene | 44 | 22 |
| (mol %) | | |
| 2/1 ratio | 4.0 | 11.2 |
| di(alkoxymethyl)naphthalene | 28 | 0 |
| (mol %) | | |
| 2,6/2,7 ratio | 11.7 | — |

### EXAMPLES 29 AND 30

10 mmol of 2-(methoxymethyl)naphthalene was reacted with 758 mmol dimethoxymethane over 4 g powdered catalyst for 4 hours at a temperature of 120° C., The results are set out in Table 8.

TABLE 8

| Example No | 29 | 30 |
|---|---|---|
| Catalyst | Beta SiO₂/ | Mordenite SiO₂/ |
| | Al₂O₃ 25 | Al₂O₃ 90 |
| 2-(methoxymethyl)naphthalene | 55 | 63 |
| conversion (mol %) | | |
| Di(methoxymethyl)naphthalene | 52 | 57 |
| (mol %) | | |
| 2,6/2,7 ratio | 8.6 | 9.7 |

### EXAMPLES 31 AND 32

10 mmol of 2-(ethoxymethyl)naphthalene was reacted with 535 mmol diethoxymethane over 4 g powdered catalyst for 4 hours at a temperature of 170° C. The results are set out in Table 9.

TABLE 9

| Example No | 31 | 32 |
|---|---|---|
| Catalyst | Beta SiO₂/ | Mordenite SiO₂/ |
| | Al₂O₃ 25 | Al₂O₃ 90 |
| 2-(ethoxymethyl)naphthalene | 21 | 34 |
| conversion (mol %) | | |
| Di(ethoxymethyl)naphthalene | 3 | 26 |
| (mol %) | | |
| 2,6/2,7 ratio | 1.4 | 2,6- only |

### EXAMPLE 33

The oxidation of 2-(ethoxymethyl)naphthalene was carried out under conditions similar to those described in U.S. Pat. No. 6,037,477 which is incorporated herein by reference. 3 mmol 2-(ethoxymethyl)naphthalene, 0.3 mmol N-hydroxyphthalimide as catalyst, 0.022 mmol Co (II) acetate tetrahydrate, 5 ml acetic acid, 1 atm O₂ at 30° C. for 20 hrs. The substrate was smoothly and efficiently oxidised at >95% conversion to ethyl-2-naphthoate (55%) and 2-naphthoic acid (38%).

### EXAMPLES 34 AND 35

Example 27 was repeated using benzene as a starting material with different catalysts. The results are set out in Table 10.

TABLE 10

| Example No | 34 | 35 |
|---|---|---|
| Catalyst | ZSM-5 (SiO₂/ | Mordenite (SiO₂/ |
| | Al₂O₃ 50) | Al₂O₃ 90) |
| Benzene conversion | 31 | 65 |
| (mol %) | | |
| Benzyl methyl ether | 19 | 25 |
| (mol %) | | |
| di(methoxymethyl) | 6 | 16 |
| benzene (mol %) | | |
| Para/ortho ratio | 71.5 | 14.4 |

### EXAMPLE 36

Example 27 was repeated using biphenyl as starting material. The only mono-substituted isomer obtained was the 4-(methoxymethyl) biphenyl and the only desired di-substituted isomer was the desired 4,4-(dimethoxymethyl)biphenyl. The results are set out in Table 11.

TABLE 11

| Example No | 36 |
|---|---|
| Catalyst | Mordenite (SiO₂/Al₂O₃ 90) |
| biphenyl conversion (mol %) | 30 |
| 4-(methoxymethyl) biphenyl | 22 |
| (mol %) | |
| 4,4′-(dimethoxymethyl) | 7 |
| biphenyl (mol %) | |
| Selectivity to desirables | 94.9 |
| (mol %) | |

### EXAMPLE 37

In order to assess the operation of the invention at a larger scale with an extrudate form of the catalyst (Mordenite (SiO₂/Al₂O₃ 200)), a 1 litre batch reactor with spinning catalyst basket was used. Methoxymethylation of 89 mmol naphthalene was carried out with 600 ml of dimethoxymethane over 45 g of extrudate catalyst (20% binder) at 110° C. for 2 hrs. The results are set out in Table 12.

TABLE 12

| Example No | 37 |
|---|---|
| Catalyst | Mordenite (SiO₂/Al₂O₃ 200) |
| naphthalene conversion | 66 |
| (mol %) | |
| (methoxymethyl)naphthalene | 40 |
| (mol %) | |
| 2/1 ratio | 6.2 |
| di(methoxymethyl)naphthalene | 19 |
| (mol %) | |
| 2,6/2,7 ratio | 11.3 |

### EXAMPLE 38

This example was carried out in order to demonstrate the reaction with reduced reagent at increased pressure without wishing to be bound by any theory, it is believed that pressure increases the concentration of dimethoxymethane in solution. Methoxymethylation of 10 mmol naphthalene was carried out with 379 mmol of dimethoxymethane over 4 g of Mordenite SiO₂/Al₂O₃ 200 at 110° C., 100 psig N₂ for 1 hr. The results are set out in Table 13.

TABLE 13

| Example No | 38 |
|---|---|
| reagent | Dimethoxymethane |
| | 379 mmol, 33.5 ml |
| naphthalene conversion | 76 |
| (mol %) | |
| (methoxymethyl)naphthalene | 43 |
| (mol %) | |
| 2/1 ratio | 5.5 |
| di(methoxymethyl)naphthalene | 22 |
| (mol %) | |
| 2,6/2,7 ratio | 10.8 |

### EXAMPLE 39

An example was carried out to show the reaction in a fixed bed operation with Mordenite SiO₂/Al₂O₃ 90 extrudate catalyst (50 ml). A feed composition of 1/18.95 mol ratio of napththalene/dimethoxymethane was used. The product composition set out in Table 14 was achieved after 63.5 hrs online with a 3.3 h residence time at 120° C. and 10 barg system pressure (N₂). The results are set out in Table 14.

TABLE 14

| Example No | 39 |
|---|---|
| Naphthalene/dimethoxymethane | 1/18.95 |
| Feed ratio (mol) | |
| naphthalene conversion | 50 |
| (mol %) | |
| (methoxymethyl)naphthalene | 31 |
| (mol %) | |
| 2/1 ratio | 3.2 |
| di(methoxymethyl)naphthalene | 7 |
| (mol %) | |
| 2,6/2,7 ratio | 6.6 |

### EXAMPLES 40 TO 42

The reaction was carried out in a continuous stirred tank operation with Mordenite SiO₂/Al₂O₃ 90 extrudate catalyst 45 g in a 1 litre autoclave equipped with spinning basket. The reactor was operated on level control at 600 ml. The feed composition had a 1/75.8 mol ratio of naphthalene/dimethoxymethane. The product compositions set out in Table 15 were achieved with a 5 h reactor residence time at 110° C.

TABLE 15

| Example No | 40 | 41 | 42 |
|---|---|---|---|
| Time (hours) | 6 | 12 | 18 |
| naphthalene conversion | 63 | 1 | 63 |
| (mol %) | | | |
| (methoxymethyl)naphthalene | 32 | 30 | 31 |
| (mol %) | | | |
| 2/1 ratio | 4.6 | 4.2 | 4.1 |
| di(methoxymethyl)naphthalene | 21 | 20 | 20 |
| (mol %) | | | |
| 2,6/2,7 ratio | 10.1 | 9.4 | 9.3 |

### EXAMPLES 43 AND 44

Benzyl methyl ether was methoxymethylated with different catalysts. The experimental conditions are identical to those in Example 27. The results are set out in Table 16.

TABLE 16

| Example No | 43 | 44 |
|---|---|---|
| Catalyst | ZSM-5 (SiO₂/ | Mordenite (SiO₂/ |
| | Al₂O₃ 50) | Al₂O₃ 90) |
| Benzyl methyl ether | 24 | 47 |
| conversion (mol %) | | |
| di(methoxymethyl) benzene | 20 | 34 |
| (mol %) | | |
| Para/ortho ratio | 123.8 | 17.0 |

1-27. (canceled)

28. A process for the alkoxyalkylation of an unsubstituted substrate or a substituted aromatic substrate, said substitution being via a carbon-carbon bond, said process comprising reacting the aromatic substrate with a dialkoxyalkane in the presence of a catalyst.

29. A process according to claim 28 wherein the alkoxyalkylation is selected for a desired substitution.

30. A process according to claim 28 wherein the catalyst is a shape selective catalyst.

31. A process according to claim 28 wherein the catalyst is non-shape selective and is used in a shape selective environment.

32. A process according to claim 31 wherein the shape selective environment includes the use of shape selective host.

33. A process according to claim 28 wherein the aromatic substrate is selected from the group consisting of a mono or polycyclic hydrocarbon compound and a mono or polycyclic heteroaryl compound.

34. A process according to claim 28 wherein the substrate is selected from the group consisting of benzene or a bi-phenyl.

35. A process according to claim 28 wherein the substrate is a polycyclic aromatic compound having fused rings.

36. A process according to claim 28 wherein the aromatic substrate is unsubstituted.

37. A process according to claim 28 wherein the aromatic substrate is mono-substituted.

38. A process according to claim 28 wherein the aromatic substrate is naphthalene, 2-(alkoxyalkyl)naphthalene, alkyl-2-naphthoate, 2-naphthoate, 2-naphthoic acid, or 2-alkylnapthalene.

39. A process according to claim 28 wherein the dialkoxyalkane is a dialkoxymethane.

40. A process according to claim 28 is a liquid-phase continuous system utilizing a fixed bed, a fluidized bed or a slurry reactor.

41. A process according to claim 40 wherein the process is carrier out in the presence of a shape selective catalyst or host.

42. A process according to claim 28 wherein a solvent is used.

43. A process according to claim 28 wherein the process is carrier out at a temperature of from about 100° C. to about 200° C.

44. A process according to claim 28 wherein the process is carried out at a pressure of from about 5 barg to about 50 barg.

45. A process according to claim 28 wherein the process is carried out for a period of from about 4 hours to about 18 hours.

46. A process for the production of a carboxylic acid or carboxylate comprising:
producing a di(alkoxyalkyl)aromatic compound or a substituted-mono(alkoxyalkyl) aromatic in accordance with the process of claim 1; and
oxidising the alkoxyalkyl side chains to the corresponding carboxylic acid or carboxylate.

47. A process for the production of a polyester comprising polymerising the carboxylic acid or carboxylate produced according to claim 46.
